(19) United States
(12) Patent Application Publication
(10) Pub. No.: US 2001/0055763 A1
(43) Pub. Date: Dec. 27, 2001

(54) INDIVIDUALLY ADDRESSABLE SOLID SURFACES FOR MULTIPLEXED OPERATIONS

(76) Inventor: SHARAT SINGH, SAN JOSE, CA (US)

Correspondence Address: BERTRAM I ROWLAND PH D, RAE VENTER LAW GROUP P C, P O BOX 60039, PALO ALTO, CA, 943060039

(21) Appl. No.: 09/466,369

(22) Filed: Dec. 15, 1999

Related U.S. Application Data
(60) Provisional application No. 60/113,853, filed on Dec. 24, 1998.

(51) Int. Cl. C12Q 1/68; C12P 19/34; G01N 33/00; C12N 9/00; C12N 1/00

(52) U.S. Cl. 435/6; 435/91.1; 435/183; 435/810; 436/94

(57) ABSTRACT

Solid moieties are provided which are individually addressable by using individual or combinations of dyes, including at least one lanthanide dye. The solid moieties may be a bulk solid having individual sites or particles, where each site can provide a unique emission spectrum defining an entity bound to the site. The individually addressable moieties may be used in multiplexed determinations for determining the presence of biopolymers or other mixture of differentiable entities. Different protocols are provided for the determinations.

### CROSS-REFERENCE TO RELATED APPLICATIONS

This application is a continuation-in-part of provisional application Ser. No. 60/113,853, filed Dec. 24, 1998, which disclosure is incorporated herein by reference.

### INTRODUCTION

1. Background

There is increasing interest in the ability to identify nucleic acid sequences, nucleotide differences from nucleic acid sequences from different sources, identification of polymorphisms, mutations and chiasmas, as well as other nucleic acid related analyses. As an increasing number of genomes from different sources are being subjected to sequencing, the ability to compare sequences has ramifications in identifying evolutionary development of species and families and identifying relationships between species. Also, by identifying fossil and progenitor DNA one can estimate time periods in the development of clades, which can be of interest to archaeologists, geologists, anthropologists, etc.

In view of the large number of nucleotides in mammalian and plant genomes, when comparing genomes it will generally be desirable to make a plurality of determinations in a single assay. The ability to perform a number of simultaneous events in a single reaction vessel with a complex DNA sample is frequently limited by erroneous events, where sequences may mismatch, there may be errors in replicating a DNA template, inherent errors in detection and the like. Besides DNA there is frequently an interest in being able to detect, desirably in a quantitative or semi-quantitative manner the level of transcription of the genes in a cellular host. Since many of the mRNAs are in low ratios to other mRNAs, techniques must be devised to be able to identify the presence of the mRNAs in lesser amounts.

Microarrays have been developed to identify sequences in genomes. However, the microarrays have many deficiencies. Their fabrication is complex and very expensive. For each new group of nucleic acid segments to be assayed, a new microarray has to be prepared. In addition, they are subject to mismatching giving false positives. Simpler, more economical and flexible methods are needed.

There is also interest in being able to identify proteins, particularly two or more proteins that complex together to form an active assembly, which has physiological significance. Where one has a complex mixture of proteins, it is of interest to determine, which proteins in the mixture form complexes and how other proteins may affect such complex formation.

2. Relevant Art

U.S. Pat. No. 5,565,324 and Still et al., Accounts of Chem.Res., 1996, 29:155 describe a combinatorial approach to the synthesis of small organic molecules using halocarbons on particles as the identifiers of the product, which halocarbons are detected by gas chromatography with flame ionization detection. U.S. Pat. No. 5,578,498 describes metal chelate containing compositions for use in chemiluminescent assays.

### SUMMARY OF THE INVENTION

Methodologies and compositions are provided using individually addressable solid moieties, particularly lanthanide coded particles in liquid arrays, where the moieties whose fluorescent dyes are imbided into the moieties include one or more fluorescent dyes, at least one being a chelated lanthanide fluorescent dye, for individual coding at individual sites, where the sites may be individual particles. The solid moieties, particularly particles, are prepared in kits for use in detecting a plurality of events occurring in the same assay mixture, where the dye labeled solid moieties may be bound with various agents, particularly nucleic acid sequences, at the surface of the moieties. The agents may be covalently conjugated to the individual solid surface. The solid moieties may be used in the determination of the presence of specific sequences in nucleic acid samples, the transcription of mRNA in cells, the presence of single nucleotide polymorphisms (snp's), the presence of mutations or other distinctive base or sequence of bases in a genome, the interaction between proteins and other entities, and the like. Various protocols may be employed with the liquid arrays and various detection schemes may be employed for deconvoluting the plurality of solid moieties resulting from the operation.

### BRIEF DESCRIPTION OF THE DRAWINGS

FIG. 1 is a schematic of an assay utilizing dethiobiotin-biotin release, where detection of a single target mRNA is realized on a microfluidic chip. Background is eliminated because decoder particles are specifically released from a hybridization sandwich.

FIG. 2 is a schematic of an assay utilizing strand displacement method, where detection of a single target mRNA is realized on a microfluidic chip. Background is eliminated because decoder particles are specifically released from a hybridization sandwich.

FIGS. 3, 4 and 5 are bar graphs of particle assays showing fluorescence of particles based on varying concentrations of the four fluorescers, 9, 10 diphenylanthracene (DPA), samarium trithenoyltrifluoroacetone bathophenanthroline (SM), europium trithenoyltrifluoroacetone bathophenanthroline (EU), and silicon phthalocyanine.

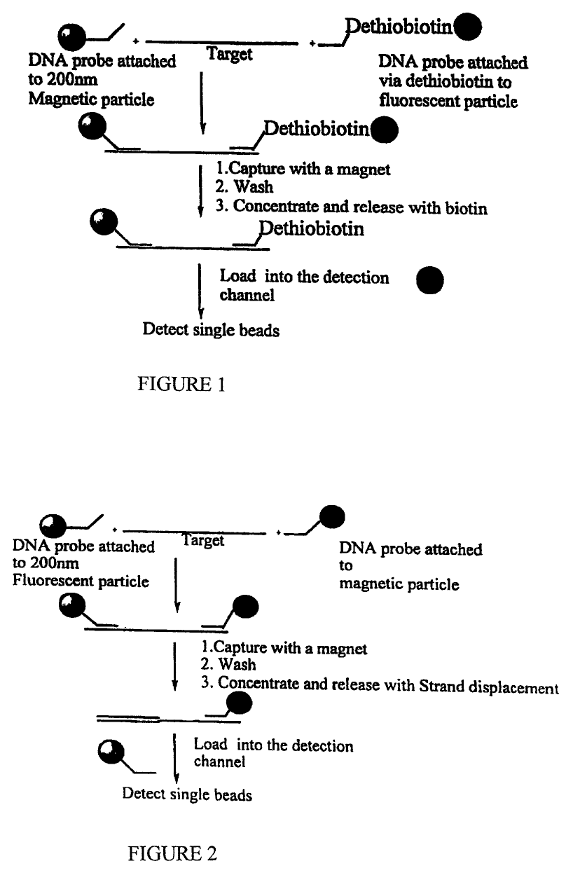

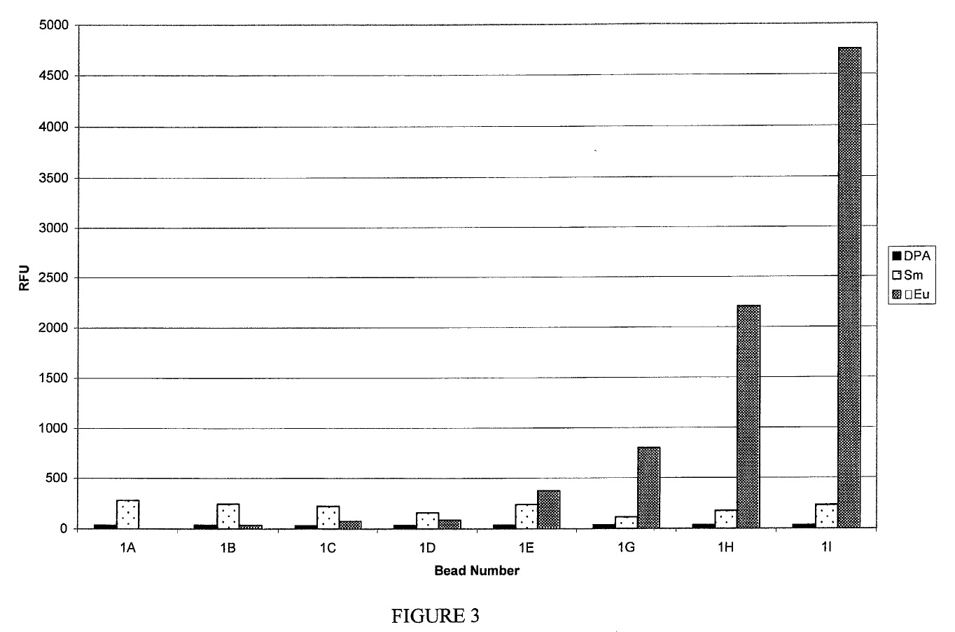

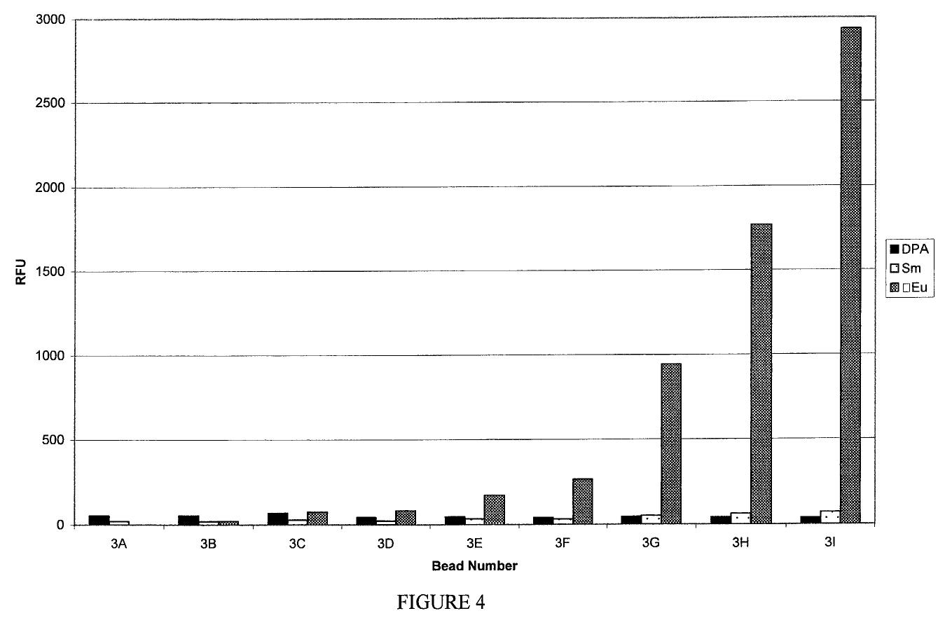

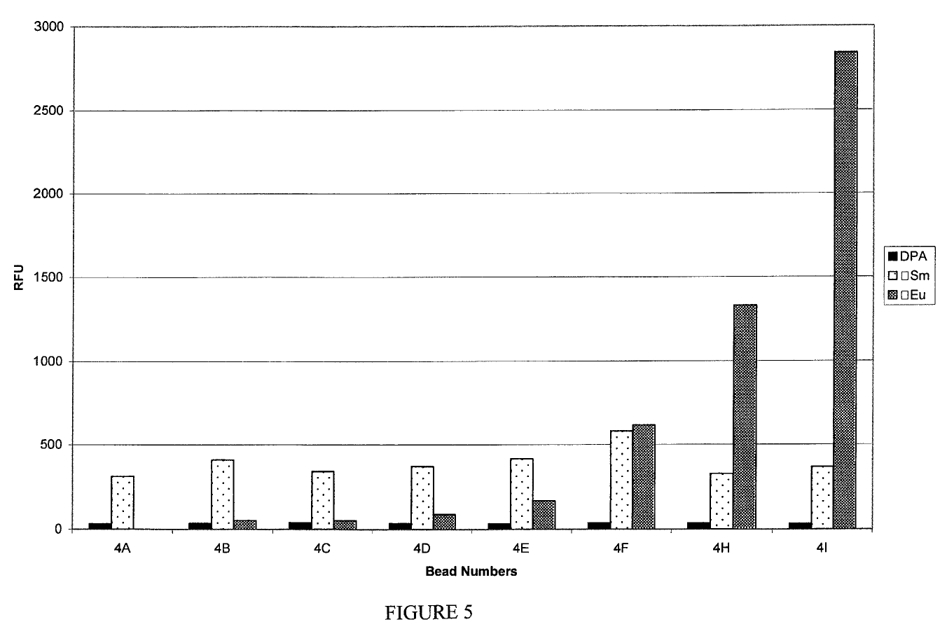

### DESCRIPTION OF THE SPECIFIC EMBODIMENTS

In accordance with the subject invention multiplexed operations may be performed, which require coding for individual events associated with the operation. Where a mixture is to be interrogated for the presence of a plurality of molecules, individually addressable moieties are employed, which are distinguished by the presence of different compositions of fluorescent compounds, where at least one of the fluorescent compounds is a fluorescent lanthanide dye. By providing a spectrum of fluorescent dye compositions, the individual moieties will have varying fluorescent emission spectra, where the spectrum may be a single wavelength or a plurality of wavelengths, contiguous or non-contiguous, which can be distinguished and related to a entity associated with the moiety. Each spectrum will differ from the spectra of other addressable moieties at at least one wavelength to be measured. The fluorescent dyes will be associated with a solid moiety, which may be a bulk support, e.g. platen, film, substrate, having the dye compositions at particular sites, or, preferably, particles of from about 5 nm to 1 mm diameter, usually in the range of about 10 to about 500 nm, which will provide individual separable sites. By dyeing the individual moieties with the appropriate fluorescent composition, the different moieties may be distinguished. By coupling the individual moieties with an entity, the emission spectrum will define the nature of the entity.

The subject moieties may be used with various complex samples in a multiplexed format to determine the presence of a plurality of target molecules being present. Compounds of particular interest include biopolymers, such as polyesters, polyamides and polyethers. The subject moieties include and may be used to detect the presence of different nucleic acid sequences, both naturally occurring and synthetic, including naturally occurring backbones and non-naturally occurring backbones, e.g. phosphates, thiophosphates, phosphoramides, amino acid amides, etc., for detecting mutations, alleles, specific DNA genomic sequences, mRNA sequences, cDNA sequences, tRNA sequences, etc. Alternatively, the subject compositions may be used to detect other biopolymer ligands, such as saccharides and poly(amino acids), using poly(amino acids) (proteins or peptides), e.g. lectins, specific binding ligands, e.g. organic molecules, both naturally occurring and synthetic of from about 125 to 5,000 Dal. The saccharides may be associated with cellular proteins, toxins, synthetic proteins, oligopeptides from various sources, and the like.

The proteins may be hormones, transcription factors, enzymes, receptors, antibodies, lectins, structural proteins, basement membrane proteins, oncogenic proteins, etc. By having a pair of molecules, which have an enhanced affinity for each other, e.g. at least 10⁶ mole⁻¹, compared to other molecules which one of the members of the pair is found in admixture, one can obtain binding between the entity and member of the pair bound to the solid moiety. Employing any of a wide variety of protocols, which have been developed and are described in the literature or future protocols, which may replace such protocols, the formation of the complex may be determined. Once can then identify the particular entity and the other member of the binding pair by the spectrum of the moiety associated with the entity.

The lanthanide dyes comprise the metals Europium (Eu), Samarium (Sm), Terbium (Th), Dysprosium (Dy), Osmium (Os), and Gadolinium (Gd), which are employed as chelates associated with energy transfer molecules. These energy transfer molecules are squarates, rubrene, phthalocyanines, Nile red, substituted naphthacenes, rhodamine derivatives, oxazines, cyanines, or the like. The energy transfer molecules may be imbibed as separate molecules or may be covalently linked to the metal chelate, preferably covalently linked. Otherwise, to obtain high efficiency of energy transfer, the energy transfer molecules should be used in at least about 3 molar excess. The dyes may be excited with a single excitation light source, particularly at wavelength 337 nm, using a nitrogen ion laser or other convenient excitation light source or a plurality of light sources at selected wavelengths.

In addition to the at least one lanthanide dye, non-lanthanide dyes may be used. Since dyes other than the lanthanide dyes can result in quenching and interfere with emission, usually not more than two non-lanthanide dyes will be used. These dyes will usually be capable of excitation at a wavelength common to the excitation wavelength for the lanthanide dyes and will emit at a wavelength other than the accompanying lanthanide dyes. In some instances, these dyes may serve as a control, serving to normalize the values obtained for the accompanying lanthanide dyes. Illustrative non-lanthanide dyes include 9, 10-diphenylanthracene, pyrene, squarene, cyanine, phthalocyanine, etc.

The lanthanide and other dyes may be associated with the solid moiety in a variety of ways: by employing a solution of the dyes whose solvent penetrates or softens the solid moiety; by linking an activated derivative of the dyes to the solid moiety; by combining the metal free chelating agent bound to the solid moiety with the lanthanide metals, or the like. For linking to the solid moiety various functionalities may be employed, such as forming an azo bond with a diazo compound and an aromatic group, e.g. benzene, an amide bond with an amino group and an acid group, where the acid group may be carboxyl, phosphoryl, sulfonyl, etc., an amino link with a carbonyl and an amine under reductive conditions, an ester group with an alcohol and an acid, ethers, using an alcohol and active halide or equivalent, etc. The chemistry for linking to a solid support is well established and need not be elaborated upon here. The functionality for forming the link may be joined to the chelating agent through a bond or chain of not more than about 36 atoms, usually not more than about 20 atoms, which are usually carbon, nitrogen, oxygen, sulfur and phosphorous, which may include functionalities in the chain as described above.

Various entities may be bound to the solid moiety, covalently or non-covalently, either directly or through a linker. The linker may be from 1 to 30 or more atoms in the chain. The linker may provide for release of the particle from the entity, using chemical, electromagnetic, catalytic or thermal means. Various chemically cleavable bonds may be employed, such as disulfides, vicinal glycols, diketones, acetals, o-nitrobenzyl ethers, etc. After performing the determination, the particles may be released from the conjugated entity and then analyzed, particularly where the conjugated entity might interfere with the isolation and identification of the particle. Where a particle is captured by duplex formation between nucleic acids, the particle may be released using denaturing conditions, such as heat, organic solvent, low ionic strength medium, etc.

The protocols for which the subject dyed solid moieties may be used will vary widely, but will be associated, for the most part, with determinations involving a plurality of determinations, where one wishes to code for a specific species in a group of like species, such as nucleic acids, including ribonucleic acids, deoxyribonucleic acids, modified nucleic acids, such as nucleoproteins, or other analogs, where the phosphate ester chain has been substituted with a different difunctional moiety from which the bases depend, e.g. amino acid; proteins, which may be involved with binding events, e.g. receptors, antibodies, enzymes, transcription factors, etc., and saccharides. Associated with the nucleic acids, may be the determination of various sequences, the determination of single nucleotide polymorphisms, the determination of transcription as mRNA or cDNA, mutations, chiasmas, inversions, repeats, etc. Associated with the proteins will be the presence of transcription factors, expression of proteins in relation to cellular status, e.g. neoplastic, stages during meiosis or mitosis, stage of differentiation, response to external stimuli, etc. Saccharides may be involved with detection of unicellular organisms, cellular status, plant cells, etc.

For investigating nucleic acids, nucleic acid sequences or their analogs will be linked to the solid moiety, where the sequence will usually having a chain length of at least about 8, more usually at least about 12, and usually not more than about 60, more usually not more than about 36 nucleotides. In some instances, there may be more than an homologous pair of sequences, for example, where bridging amplification is used. In this instance there will be a 5′-3′ sequence homologous with one sequence of a target sequence and a 3′-5 sequence, which will be the same as a sequence in the target sequence spaced apart from the first sequence in the 5′-3′ direction. See, for example, U.S. Pat. No. 5,641,658. The emission spectrum of the dyes will indicate what the sequences bonded to the solid support are and, to that extent, the target sequence. Alternatively, one may use the PCR reaction for amplifying sample DNA associated with a specific primer. Other techniques may also be used for amplification, such as cloning, NABSA, SDA, isothermal amplification, etc.

Where one is interested in determining whether there are certain sequences in a sample, for example, for diagnosing the presence of foreign cells in a mammalian sample, the transcription pattern of cells or other interest in the presence of an extended sequence, greater than about 30 nucleotides, the solid moiety can be coded for the sequence, which hybridizes to the target sequence. The sample is combined with the solid moiety under hybridizing conditions, whereby the target DNA will hybridize to an homologous sequence (the hybridizing sequence may differ, usually by not more than about 10% of the total number of bases, involving insertions, deletions, transitions and transversions), preferably a complementary sequence, under appropriate hybridization conditions, particularly during the wash stage, where non-specific nucleic acid is removed. If desired, the bound sequence could be provided with a photoactivated cross-linking agent, so that after the hybridization, the cross-linking agent would be photoactivated, and the target sequence would be covalently bonded to the solid moiety.

In order to ascertain the presence of the target sequence bound to the support, there are many protocols, which may be used. If particles are employed, one may label the target sequence with a ligand, so that only those particles to which the target sequence is bound would be captured by the receptor for the ligand. For example, during amplification of a nucleic acid sequence, the primer may be labeled with biotin or other small organic ligand for which a receptor is available. When the target sequence binds to the homologous sequence bound to the dyed particle, the particles to which the target sequence is bound may be sequestered with streptavidin bound to a solid support. The particles may then be individually irradiated and the fluorescence analyzed.

For determining nucleic acids, usually a nucleic acid sample will be processed prior to being used with the particles. Processing may include isolation, purification, fragment formation with restriction endonucleases, amplification with PCR or other technique, denaturation, fusion with primers, attachment to various entities, such as ligands, labels, chelating agents, etc. Depending upon the prior processing, the medium in which the DNA is present may be replaced with a different medium for hybridizing. Once the DNA sample is prepared, it is combined with the particles under hybridizing conditions. Where one is interested in the presence of a sequence, such as identifying an allele, a unicellular organism, e.g. prokaryotic, fungal, protista, etc., mutation, e.g. excision, insertion, transversion or transition, the probe on the particles will be specific for the DNA sequence of interest. By having a ligand on the sample DNA, the duplex of sample DNA and probe will bind to a receptor for the ligand. The receptor may be bound to a surface, such as another particle, e.g. a magnetic particle, a solid support, or other means that allows for sequestering those particles bound to sample DNA from particles that are not bound to sample DNA. One may then isolate the particles and determine their emission spectrum, which will define the probe bound to the particles and identify the DNA sequence.

Where one wishes to reduce the possibility of detecting a homologous sequence as distinct from a complementary sequence, one may use strand displacement, where the ssDNA will bind to the sample DNA releasing the particle. For strand displacement, the target nucleic acid has a ligand for binding to a receptor, which is bound to a solid support. After capturing the duplexed nucleic acid, one can release particles specifically by adding ssDNA having the desired sequence complementary to the target sequence of interest. This will result in specific release of particles that are bound to the particles, while leaving sequences that are bound and have differences from the target sequence. One may then isolate the invididual particles that are released. Again, one will determine the spectrum of the particles to identify the sample DNA.

Instead of determining a sequence, one may be interested in determining single nucleotide polymorphisms (snp's), particularly where the determination is multiplexed. The subject particles allow for high multiplicities of snp determinations in a single reaction vessel. The processed sample would be combined with the particles having bound hybridizing sequences present on the particles, where the emission spectrum of the particles would indicate the sample sequence binding to the particles. In this case, the sample DNA may not be labeled. One would then add probes, which bind to the sample DNA at a site, which upon single nucleotide elongation of the probe, the nucleotide will be complementary to the nucleotide at the putative snp site. By using labeled terminating nucleotide triphosphates, the additional nucleotide will represent whether a snp is present. One may have four reaction vessels, where the particles are associated with snps having the same base or one may have fewer than four reaction vessels, where each terminating nucleotide has a different label for the individual bases. Particularly, one may have different ligands, so that probes bound to sample DNA duplexed to particles would be captured at different sites and be segregated from each other. By releasing the particles and identifying their individual spectra, one would know which sequence had which nucleotide at the putative snp site.

Instead of single nucleotide extension, one could provide for ligation, by having a second primer in solution, where the two primers are separated by the snp. Where the snp is present, the two primers would be ligated and the second primer could allow for selection of those particles where ligation occurred. Other means for providing a moiety to be bound to the particle that allows for selection of the particles may also be employed.

One may use the particles in heterogeneous assays, where the particles sere as labels to identify a plurality of proteins in a sample. For example, when assaying for the status of a cell at various stage of mitosis, in the case of cancer or precancer, cells under stress, or the like, the individual particles would be associated with individual monoclonal antibodies. Various protocols may be used to identify the proteins. The protein fraction of the cells could be spread on a surface to which the proteins would strongly bind. The binding could be specific, where monoclonal antibodies are arrayed on the surface, where the bound monoclonal antibodies bind to an epitope different from the epitope to which the monoclonal antibodies conjugated to the particles binds. One can monitor the activity of cells, using intact cells, either fixed or alive, or cell lysates. By having the particles conjugated with ligands, proteins that bind to surface membrane proteins or antibodies that bind to surface membrane proteins, one can detect the presence of a particular target molecule present on the surface of the cell or internal to the cell. Thus, one can simultaneously monitor the changes in expression in a cell and graph the variations of different proteins simultaneously. By stimulating the cells in various manners, once can obtain a profile of the changes in expression with time as a result of the stimulus.

The particles may be isolated in a variety of ways, using panning, e.g. using pans with dimples and vacuum to pull the particles into individual dimples, fluorescence activated cell sorter, capillary electrophoresis, or the like. Individual particles may then be irradiated and the spectrum analyzed. Generally not more than six different wavelengths will be detectable, usually not more than about four different wavelengths. The spectrum should provide a differentiation between concentrations of at least about 100 RFU, preferably about 200 RFU. Peak heights may be as high as 10,000 RFU or more, frequently not exceeding 5,000 RFU. Time delayed emission maxima may be used for decoding the particle. A commercially available filter wheel may be used in the detection, while a single excitation source is employed, e.g. nitrogen ion laser at 337 nm.

The particles may be dyed using the appropriate lanthanide dye mixture, the particles and a solvent which able to soften the particles. For particles made of latex or other similar organic polymer, e.g. poly(methyl methacrylate), polystyrene, polyethylene, polypropylene, poly(vinyl ethyl ether), etc., about 10 to 50 vol. % of ether substituted alkanols boiling above 100° C. are used in an aqueous medium at temperatures in the range of about 85-950C. The lanthanide dye mixture is dissolved in the solution and the reaction allowed to proceed for about 1-10 minutes, depending upon the volume, concentration of the dyes, nature of the particles and solvent, desired level of dyeing of the particles and the like. The particles may then be isolated, washed with a lower alkanol, e.g. ethanol, and then agitated thoroughly in a milk alkaline solution.

In carrying out the multiplexed determinations, usually there will be at least about 5 different moieties employed, more usually at least about 10 different moieties, and not more than about 5×10⁵, usually not more than about 5×10⁴, moieties. The number of moieties which may be employed will depend upon the nature of the determination, whether the moieties are sites of a solid support or individual particles, sensitivity of discrimination of fluorescence emission, whether one is performing PCR or other process with the particles, the complexity of the sample, and the like. The particles may be magnetic particles or diamagnetic particles. Kits can be provided where from 5 to 10⁶ or more moieties may be provided, as particles or bulk solid supports.

As illustrative of the subject methodology, a microfluidic-based card device designed for a capture-release protocol is employed. The sample is mixed with magnetic particles and introduced into the sample reservoir of the device. The sample members have a ligand label for binding to a receptor. Targets bind to their complementary members on the particles. The contents of the reservoir are then transported by electrokinesis to a magnetic zone, where the magnetic particles are captured and washed free of non-specifically bound components of the sample. The particles are then transported to a site where the receptor bound and particles to which sample is bound are captured. The remaining particles are removed from the site. Where the sample is a nucleic acid sample, with a multiplicity of target sequences, particles may be sequentially removed from the receptor site using strand displacement, by transporting specific nucleic acid strands to the site and incubating, where the particles having the specific sequence are sequentially released and read or preferably, mixtures of sequences are transported to the site, and groups of particles are released and analyzed individually.

The following examples are offered by way of illustration and not by way of limitation.

### EXPERIMENTAL

### Dyeing of Particles With Three Fluorescent Dyes

Materials

Particles: Seradyn Uniform Microparticles catalog # 1300-2120 (mfg lot #432 VP2, pkg lot # 1485),

2-Ethoxyethanol: Aldrich catalog #25,637-4 (spectrophotometric grade)

Water: Deionized by Millipore's Milli-Q system

9,10-Diphenylanthracene: Aldrich catalog #D20,500-1 (purity 97%)

Samarium trithenoyltrifluoroacetone bathophenanthroline: prepared in-house

Europium trithenoyltrifluoroacetone bathophenanthroline: prepared in-house

Ethanol: EM Science, anhydrous (suitable for Histology)

Silicon (IV) phthalocyanine bis (trihexylsilyloxide)—Aldrich catalog #42,815-9

Synthesis

To each 10 ml round bottom flask is added 250 μl particles in 250 μl 2-ethoxyethanol and 500 μl water and the particle suspensions are stirred and heated in a 94° C. oil bath for 5 minutes.

A mixture of 1 μl of 1 mg/ml diphenylanthracene and/or silicon (IV) phthalocyanine bis (trihexylsilyloxide), Sm (TTA),DPP (the same concentration per assay set; 12.5 μl, 25 μl, 50 μl, 100 μl of 50 mg/ml) and Eu (TTA),DPP (0, 10, 20, 40, 80, 160 μl of 1 mg/ml and 32, 64, 128 μl of 10 mg/ml in each assay set) in 2-ethoxyethanol is added to each particle suspension. The reaction mixtures are heated in a 940C oil bath for 5 minutes.

The resulting particle suspensions are transferred from the 10 ml round bottom flask to 1.5 ml Eppendorf tubes, then centrifuged at 14,000rpm for approximately 5 minutes.

The reaction solvents are drawn off, a 1 ml ethanol wash added and the particle suspensions are sonicated for 30 min.

These sonicated suspensions are centrifuged at 14,000rpm for approximately 5 minutes. The ethanol is drawn off, 1 ml 0.5 N NaOH is added to each tube and the mixtures are again sonicated for 30 min.

Analysis

Each set of particles is assayed using the Hitachi F4600 Fluorometer, with single excitation wavelength of 360 nm. Sample preparation for analysis involves the dilution of 10 μl of each particle suspension into 95 μl water, then from this dilution 5 μl is diluted into 990 μl water for analysis. RFU's for DPA, Eu(TTA),DPP, Sm(TTA),DPP and silicon (IV) phthalocyanine bis (trihexylsilyloxide) are recorded at 410.0, 610.0, 645.4 and 690.0 nm emission wavelengths, respectively.

### PREPARATION OF IN-HOUSE REAGENTS

Materials

Thenoyltrifluoroacetone: Adlrich catalog # T2,700-6 (purity 99%)

Samarium trichloride hexahydrate: Aldrich catalog #24,880-0 (purity 99+%)

Europium trichloride hexahydrate: Aldrich catalog # 21,288-1 (purity 99.9%)

Bathophenanthroline: Fluka catalog 3 11880 (purity 99+%)

Sodium hydroxide particles: Aldrich catalog # 36,717-6 (20-40 mesh, purity 97%)

Ethanol: EM Science, anhydrous (suitable for Histology)

Toluene: Aldrich catalog # 24,451-1 (purity 99.8%)

Synthesis

1. In a 100 ml round bottom flask was dissolved 2.00 gm thenoyltrifluoroacetone (9.00 mmoles, 3 equiv.) in 15 ml ethanol.

2. 0.36 gm sodium hydroxide particles (9.00 mmoles, 3 equiv.) were added and the reaction mixture was stirred and heated at 40° C. for 30 minutes.

3. To the reaction mixture was added 1.09 gm samarium (or europium) trichloride hexahydrate (3.00 mmoles, 1 equiv.) and stirring and heating of the reaction mixture was continued for one hour.

4. First the solvent was removed in vacuo, then sodium chloride was removed by dissolving the entire solid in hot toluene and filtration.

5. To the filtrate was added 1.0 gm bathophenanthroline (3.00 mmoles, 1 equiv.) and the reaction mixture was run on a rotary evaporator for 1½ hours, replenishing the toluene throughout the reaction time and removing it at the end of the reaction time.

Europium trithenoyltrifluoroacetone bathophenanthroline [Eu (TTA),DPP]: same procedure as above, substituting europium trichloride hexahydrate for samarium trichloride hexahydrate.

PARTICLE DYEING REAGENT CHART

| | | | CON- | | |
|---|---|---|---|---|---|
| | | | CEN- | | |
| | | | TRATION | VOLUME | |
| ASSAY | VOLUME | VOLUME | OF Eu | OF Eu | |
| # | OF | OF Sm | (TTA) | (TTA) | VOLUME |
| (BEAD | DPA | (TTA) | 3TPP | 3TPP | OF 2- |
| #) | (ul) | 3DPP | (ul) | (ul) | Ethoxyethanol |
| 1A | 1 | 50 | 1 | 0 | 195 |
| 1B | 1 | 50 | 1 | 10 | 185 |
| 1C | 1 | 50 | 1 | 20 | 175 |
| 1D | 1 | 50 | 1 | 40 | 155 |
| 1E | 1 | 50 | 1 | 80 | 115 |
| 1G | 1 | 50 | 10 | 32 | 183 |
| 1H | 1 | 50 | 10 | 64 | 131 |
| 1I | 1 | 50 | 10 | 128 | 67 |
| 3A | 1 | 12.5 | 1 | 0 | 232.5 |
| 3B | 1 | 12.5 | 1 | 10 | 222.5 |
| 3C | 1 | 12.5 | 1 | 20 | 212.5 |
| 3D | 1 | 12.5 | 1 | 40 | 192.5 |
| 3E | 1 | 12.5 | 1 | 80 | 152.5 |
| 3F | 1 | 12.5 | 1 | 160 | 72.5 |
| 3G | 1 | 12.5 | 10 | 32 | 220.5 |
| 3H | 1 | 12.5 | 10 | 64 | 168.5 |
| 3I | 1 | 12.5 | 10 | 128 | 104.5 |
| 4A | 1 | 100 | 1 | 0 | 145 |
| 4B | 1 | 100 | 1 | 10 | 135 |
| 4C | 1 | 100 | 1 | 20 | 125 |
| 4D | 1 | 100 | 1 | 40 | 105 |
| 4E | 1 | 100 | 1 | 80 | 65 |
| 4F | 1 | 100 | 10 | 16 | 129 |
| 4H | 1 | 100 | 10 | 64 | 64 |
| 4I | 1 | 100 | 10 | 128 | 128 |

It is evident from the above results and the accompanying figures that the subject particles allow for multiplexing various determinations and operations, where one can distinguish events occurring in relation to the particles by identifying the emission spectrum of the particle. Of course only individual points need be determined and by using the isobiestic point for two or more combinations of dyes, one has a value to normalize the other values.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. In a method for performing a multiplexed determination on a surface of a solid moiety comprising independently addressable sites, the improvement which comprises:
employing mixtures of lanthanide dyes for encoding said moieties in said solid moiety; and
decoding said sites by their emission spectra.

2. A method according to claim 1, wherein said lanthanide dyes are chelates of Eu, Sm, Tb, and Dy.

3. A method according to claim 1, wherein said lanthanide dyes code for a nucleic acid sequence bound to said moieties.

4. A method according to claim 3, wherein said lanthanide dyes are chelates of Eu, Sm, Tb, and Dy

5. A method according to claim 1, wherein said moieties comprise from 0 to 2 fluorescent dyes other than lanthanide dyes having an absorption wavelength overlapping the absorption wavelength of at least one of said lanthanide dyes.

6. A method according to claim 1, wherein said moieties are particles of an average diameter in the range of about 5 nm to 1 mm.

7. A method for determining the presence of at least 5 different nucleic acid sequences in a sample comprising a mixture of nucleic acid molecules, said method comprising:
combining independently addressable particles with said sample, wherein said independently addressable particles are characterized by comprising a dye composition consisting of at least one lanthanide dye and from 0 to 2 fluorescent dyes other than lanthanide dyes, having an absorption wavelength overlapping the absorption wavelength of at least one of said lanthanide dyes, having nucleic acid oligomers bound to the surface of said particles, wherein the sequence of said oligomers bound to said particle is related
to the emission spectrum of said dye composition of said particle under hybridization conditions;
separating particles to which sample nucleic acid molecules have hybridized; and
identifying the sequence of said nucleic acid molecules bound to said particles by the emission spectra of said particles.

8. A method according to claim 7, including the additional step of amplifying nucleic acid molecules or portions thereof in said sample.

9. A method for identifying at least 5 single nucleotide polymorphisms in a sample comprising a mixture of nucleic acid molecules, said method comprising:
combining independently addressable particles with said sample, wherein said independently addressable particles are characterized by comprising a dye composition consisting of at least one lanthanide dye and from 0 to 2 fluorescent dyes other than lanthanide dyes, having an absorption wavelength overlapping the absorption wavelength of at least one of said lanthanide dyes, having nucleic acid oligomers bound to the surface of said particles, wherein said oligomers bound to each particle hybridize to a nucleic acid molecule comprising a single nucleotide polymorphism;
extending said oligomers with a template dependent DNA polymerase, wherein extension requires the presence of said single nucleotide polymorphism;
separating particles having extended oligomers; and
determining the single nucleotide polymorphisms by determining the emission spectrum of each of said particles.

10. A kit comprising a plurality of independently addressable solid surfaces, wherein said independently addressable particles are characterized by comprising a dye composition consisting of at least one lanthanide dye and from 0 to 2 fluorescent dyes
other than lanthanide dyes, having an absorption wavelength overlapping the absorption wavelength of at least one of said lanthanide dyes.

11. A kit according to claim 10, having at least 10 different independently addressable solid surfaces.

12. A kit according to claim 11, wherein said independently addressable solid surfaces are particles.

13. A kit according to claim 10, wherein said lanthanide dyes are chelates of Eu, Sm, Tb, and Dy.

14. A kit according to claim 13, having from 1 to 2 fluorescent dyes other than lanthanide dyes, having an absorption wavelength overlapping the absorption wavelength of at least one of said lanthanide dyes.

15. A kit according to claim 10, wherein to each of said independently addressable surfaces is bound a different DNA oligomer, wherein the emission spectrum of each surface defines the sequence of the oligomer bound to said surface.

16. A kit according to claim 15, wherein said surfaces are particles.

17. A kit according to claim 10, wherein to each of said independently addressable surfaces is bound a poly(amino acid), wherein the emission spectrum of each surface defines the sequence of poly(amino acid) bound to said surface.

18. A kit according to claim 17, wherein said surfaces are particles.
